(19) United States
(12) Patent Application Publication
(10) Pub. No.: US 2008/0108546 A1
(43) Pub. Date: May 8, 2008

(54) Novel Human Card-Only Protein That Inhibits Pro-Il-1 Beta Maturation

(75) Inventors: Peter Vandenabeele, Sint-Amandsberg (BE); Mohamed Lamkanfi, Ghent (BE); Geertrui Denecker, Sint-Amandsberg (BE)

Correspondence Address: TRASK BRITT, P.O. BOX 2550, SALT LAKE CITY, UT, 84110, US

(73) Assignees: VLAAMS INTERUNIVERSITAIR INSTITUUT, ZWIJNAARDE BELGIUM (BE); UNIVERSITEIT GENT, GENT BELGIUM (BE)

(21) Appl. No.: 11/597,699

(22) PCT Filed: Dec. 15, 2005
(86) PCT No.: PCT/EP05/52401

§ 371 Date: Sep. 28, 2007

(30) Foreign Application Priority Data
May 27, 2004 (EP) 04102359.9

(51) Int. Cl. A61K 38/00; A61P 29/00; C07H 19/00; C07K 14/47; C12N 9/00

(52) U.S. Cl. 514/2; 435/183; 530/351; 536/22.1

(57) ABSTRACT

The present invention relates to a novel member of the card protein family. More specifically, it relates to a novel human card-only protein. The invention relates further to the use of this protein to inhibit pro-interleukin-1β maturation, preferably without inducing NF-_{κ}B activity or apoptosis.

The present invention relates to a novel member of the card protein family. More specifically, it relates to a novel human card-only protein. The invention relates further to the use of this protein to inhibit pro-interleukin-1β maturation, preferably without inducing NF-κB activity.

Interleukin-1β (IL-1β) has been implicated in a wide variety of inflammatory conditions in vivo (reviewed in Dinarello et al., 1993). The processing of inactive pro-IL-1β into its biologically active form is absolutely dependent on caspase-1, a prototypical member of a conserved family of cysteine proteases that specifically cleave after aspartic acid residues. Caspase-1 plays a key role in inflammatory responses by cleaving pro-IL-1β and pro-IL-18 into secreted pro-inflammatory cytokines (Cerretti et al., 1992; Ghayur et al., 1997; Gu et al., 1997). Experiments involving caspase-1 deficient mice have provided firm evidence for an important role for this protease in pro-inflammatory responses (Kuida et al., 1995). For example, caspase-1 deficient mice display marked resistance to endotoxic shock following challenge with high doses of lipopolysaccharide (LPS) due to a failure in the production of the pro-inflammatory cytokines IL-1β and IL-18. Recently it has been discovered that the latter cytokines are matured in a large procaspase-1-containing protein complex, called the ‘inflammasome’ (Martinon et al., 2002). Procaspase-1 is recruited to this complex through its N-terminal caspase recruitment domain (CARD). This protein module of approximately 100 amino acids in length is a homotypic oligomerization domain shown to be involved in the assembly of protein platforms that promote proteolytic activation of recruited caspases in the context of apoptosis and inflammation.

ICEBERG and COP/Pseudo-ICE are two human-specific CARD-only proteins that share a high degree of sequence homology to the prodomain of procaspase-1, reaching 93% and 73% respectively (Druilhe et al., 2001; Humke et al., 2000; Lee et al., 2001). Both ICEBERG and COP/Pseudo-ICE are encoded by caspase-like genes that have acquired premature nonsense mutations leading to the production of essentially CARD-only molecules. Interestingly, their genes are mapped to chromosome 11q22, adjacent to the procaspase-1 gene and have probably arisen by a recent gene duplication event. Both proteins bind to and prevent procaspase-1 activation and the subsequent generation of IL-1β (Druilhe et al., 2001; Humke et al., 2000; Lee et al., 2001). However, in contrast to ICEBERG, COP/Pseudo-ICE also interacts with RIP2 in a CARD-CARD interaction, and activates the transcription factor NF-κB (Druilhe et al., 2001; Humke et al., 2000).

Using bioinformatics approaches, we have identified a human gene that encodes a novel CARD-containing protein, which we termed INCA (Inhibitory Card). Similar to ICEBERG and COP/Pseudo-ICE, the INCA protein is relatively short (110 amino acids), composed essentially of only a CARD domain that shares 81% sequence identity with the prodomain of procaspase-1. Said INCA protein has been disclosed in WO0198468, where it was described as a protease. However, the gene encoding the protein has never been isolated. Moreover, surprisingly we demonstrated that INCA doesn't show protease activity, but binds to procaspase-1 and inhibits caspase-1-induced proIL-1β maturation and release. Like ICEBERG, but in contrast to COP/Pseudo-ICE and the prodomain of procaspase-1, INCA does not bind to RIP2 and its overexpression does not induce NF-κB activation.

A first aspect of the invention is a genomic nucleic acid sequence, encoding a CARD only protein, comprising SEQ ID NO 3. Preferably, said genomic sequence is essentially consisting of SEQ ID NO 3, more preferably said genomic sequence is consisting of SEQ ID NO 3. Said genomic sequence is encoding a CARD only protein comprising SEQ ID NO 2. Said genomic sequence may be used, as a non-limiting example, to screen for mutations in the gene. Such mutations would lead to a stimulation of the inflammasome complex and may be important in chronic inflammation.

Another aspect of the invention is the use of a CARD only protein, comprising SEQ ID NO 2, or a functional fragment thereof, to inhibit caspase-1 activity. Still another aspect of the invention is the use of a CARD only protein, comprising SEQ ID NO 2, or a functional fragment thereof, to inhibit pro-interleukin-1β maturation. A functional fragment as defined here is a fragment that is still capable of inhibiting caspase-1 activity and/or inhibiting pro-interleukin-1β maturation. A non-limiting example of such fragment is amino acid 1-89 of SEQ ID NO 2. Another non-limiting example of such a fragment is amino acid 27-83 of SEQ ID NO 2. Alternatively, based on the INCA sequence, peptidomimetic compounds may be designed that inhibit caspase-1 activity. Such an inhibition can be useful to treat inflammation. Preferably said CARD only protein is essentially consisting of SEQ ID NO 2, more preferably said CARD only protein is consisting of SEQ ID NO 2. In a preferred embodiment, said inhibition of caspase-1 activity and/or pro-interleukin-1β maturation is not accompanied with NF-κB induction. In another preferred embodiment, said inhibition of caspase-1 activity and/or pro-interleukin-1β maturation is not accompanied with apoptosis.

As INCA exerts its inhibiting action by interacting with the prodomain of procaspase-1, it is clear for the person skilled in the art that the inhibiting activity can be counteracted by inhibiting this interaction. Inhibition of said interaction can be realized in several ways. As non-limiting examples, antibodies may be generated against the CARD, or against the CARD binding domain of the interaction partner. Alternatively, CARD derived mutants or fragments that interfere with the interaction can be used.

### BRIEF DESCRIPTION OF THE FIGURES

FIG. 1: Gene organization, transcript and protein sequences of INCA. (A) Organization of caspase-12, caspase-4, caspase-5, caspase-1, COP, INCA and ICEBERG genes on human chromosome 11q22. (B) Nucleotide sequence of the INCA cDNA. The start and stop codons are indicated in bold letters. The positions of intron/exon borders are indicated by inverted triangles. (C) A schematic structure of the INCA gene showing the intron/exon borders. Consensus splice donor (GT) and acceptor (AG) motifs are underlined and the length of the introns is indicated in base pairs (bp). The start and stop codons are shown in bold letters. (D) An amino acid sequence alignment of INCA, COP/Pseudo-ICE, ICEBERG and the first 110 residues of procaspase-1. Black and white boxes indicate identical and non-identical amino acids, respectively. Residue position numbers are indicated on the right. (E) Schematic representation of the CARD-proteins depicted in (A). The CARD and caspase domain modules are indicated with an arrow and are drawn to scale. The molecular mass (M_{w}) of the proteins is indicated in kDa.

FIG. 2: Tissue distribution of INCA mRNA expression. The expression of procaspase-1 (CASP1) and INCA mRNAs in 22 adult and 2 fetal human tissues and in the human HeLa cell line was determined by RT-PCR. cDNAs were amplified using specific primers for procaspase-1, INCA or β-actin. The respective, resulting PCR products were analyzed by agarose gel electrophoresis and visualized by ethidium bromide staining. Fragment size is indicated in kbp. The identity of the procaspase-1 and INCA PCR products was confirmed by DNA sequencing.

FIG. 3: INCA expression is Upregulated by IFN-γ in THP-1 and U937 cells. The expression of procaspase-1 (CASP1) and INCA mRNA in differentially stimulated human THP-1 (A) and U937 (B) cells was determined by RT-PCR. THP-1 cells were seeded at 4.10⁵ cells/ml and U937 cells at 2.10⁵ cells/ml. After 36 h, cells were left untreated or stimulated with LPS (1 μg/ml), human TNF-α (1000 IU/ml), human IFN-γ (1000 IU/ml) or combinations of these stimuli for an additional 12 h. Total RNA was isolated and cDNAs were amplified using specific primers for procaspase-1, INCA or β-actin. The respective, resulting PCR products were analyzed by agarose gel electrophoresis and visualized by ethidium bromide staining. PCR fragment size is indicated in kbp. The identity of the procaspase-1 and INCA PCR products was confirmed by DNA sequencing.

FIG. 4: Interactions of INCA with other CARD-containing proteins. Co-immunoprecipitation assays were performed using lysates from 293T cells that have been transiently transfected with plasmids encoding various epitope-tagged proteins as indicated, including Flag-INCA, E-INCA, E-procaspase-1, E-COP, E-ICEBERG, E-RIP2 and E-procaspase-2 CARD. Immunoprecipitates were prepared using anti-Flag antibody adsorbed to protein G-sepharose and analyzed by SDS-PAGE/immunoblotting using anti-E epitope tag antibody and chemoluminescence-based detection. Aliquots of the same lysates were also analyzed directly by SDS-PAGE/immunoblotting as indicated. IP, immunoprecipitation; WB: Western blotting.

FIG. 5: INCA does not induce NF-κB activation. (A) 293T cells were transiently cotransfected with a NF-κB dependent luciferase reporter and the indicated amounts of plasmids encoding procaspase-1 C285A, COP/Pseudo-ICE, INCA or ICEBERG. Total DNA was maintained at 0.7 μg by the addition of control plasmid DNA. 24 h after transfection, lysates were analyzed for NF-κB activity as described in Materials and Methods. (B) Aliquots of the same whole cell lysates were analyzed by SDS-PAGE/immunoblotting to confirm the appropriate expression of all constructs. Data represent the mean ±S.D. (n=3).

FIG. 6: INCA does not inhibit NF-κB activation by TNF, procaspase-1 C285A, COP/Pseudo-ICE or RIP2. (A) 293T cells were transiently cotransfected with a plasmid allowing NF-κB dependent luciferase reporter expression, 0.2 μg of a plasmid encoding either procaspase-1 C285A, COP/Pseudo-ICE or RIP2 and 0.6 μg of a plasmid coding for INCA, ICEBERG or IKK-β DN. In another setup, cells were transiently co-transfected with a plasmid allowing NF-κB dependent luciferase expression and 0.6 μg of a plasmid encoding either INCA, ICEBERG or IKK-β DN and treated with 500 IU/ml human TNF for induction of NF-κB activation. Total DNA was maintained at 1 μg by the addition of control plasmid DNA. 24 h after transfection, lysates were analyzed for NF-κB activity as described in Materials and Methods. (B) Aliquots of the same whole cell lysates were analyzed by SDS-PAGE/immunoblotting to confirm the appropriated expression of all constructs. Data represent the mean ±S.D. (n=3).

FIG. 7: INCA inhibits LPS-induced release of IL-1β. THP-1 cells were infected using a retroviral vector encoding Flag-tagged COP/Pseudo-ICE or INCA and a neomycin-resistance gene. After selection with neomycin antibiotic, stable transfectant THP-1 mass cultures were assayed for the expression of procaspase-1, COP/Pseudo-ICE and INCA using an antibody against caspase-1 CARD that is cross-reactive with the three proteins (A). Expression of the Flag-tagged proteins by was re-verified using anti-Flag antibody (not shown). Control and transfected THP-1 cells were treated with or without 0.1 μg/ml LPS (B) or 10 μg/ml LPS (C). Following 48 h treatment, supernatants were collected and IL-1β concentrations were determined. Data represent the mean ±S.D. (n=3).

### EXAMPLES

### Materials and Methods to the Examples

Isolation of INCA cDNA

A genomic sequence containing a yet unidentified CARD domain was identified by searching the GenBank™ High Throughput Genomic Sequence (HTGS) database for sequences similar to the prodomain of procaspase-1 using the BLASTn program. This gene, which we named INCA (inhibitory CARD), was present in four different clones of the HTGS database (GenBank accession numbers AP002787, AC027011, AP001024, AC021452). A hypothetical INCA cDNA sequence was assembled using several bioinformatics programs. Subsequently, the predicted INCA cDNA sequence was amplified by PCR from different human tissues and cell lines using 5′-CGAGGAGGGATCCTAGCCATGGCCGACAAGGTCCTGAAGGAG3′ (INCA-forward) and 5′-TGAACTCTCGAGAACCTAGGAAGGAAGTACTATTTGAG-3′ (INCA-REVERSE) as primers. INCA cDNA sequences were cloned into pCAGGS and sequenced, confirming the in silico prediction.

RNA isolation and Reverse Transcriptase-Polymerase Chain Reaction (RT-PCR)

HeLa cells and the human monocytic cell lines U937 and THP-1 were cultured according to supplier's instructions. THP-1 cells were seeded at 400 000 cells/ml medium and U937 cells at 200 000 cells/ml medium in a 6-well plate. After 36 h, the cells were either left untreated or stimulated with LPS (1 μg/ml), human TNF-α (1000 IU/ml), human IFN-γ (1000 IU/ml) or combinations of these stimuli for an additional 12 h. Total RNA was isolated from cells with the RNeasy isolation kit (Qiagen, Hilden, Germany). First strand cDNA libraries were made according to instructions with the SuperScript PreAmplification system (Invitrogen, Carlsbad, Calif., USA). Levels of RNA were normalized using UV-spectrophotometry at 260 nm wavelength and β-actin specific control primers. For RT-PCR analysis of INCA mRNA, cDNA samples derived from multiple human adult tissues (OriGene Technologies, Rockville, Md., USA) were amplified using INCA-specific primers (5′-GGATCCTAGCCATGGCCGACAAGGTCCTGAAGGAG-3′, (INCA-forward) and 5′-TGAACTCTCGAGAACCTAGGAAGGAAGTACTATTTGAG-3′, (INCA-reverse). The resulting PCR products were size-fractionated by electrophoresis in 1.5% agarose gels, then stained with ethidium bromide for UV-photography. In order to control the amplified product, the amplified band was excised from gels, purified and sequenced.

Expression Plasmids

The following expression plasmids were obtained from the indicated sources: pNF-conLuc, encoding the luciferase reporter gene driven by a minimal NF-B responsive promoter was a generous gift from Dr. A. Israël (Institut Pasteur, Paris, France). The plasmid pUT651, encoding β-galactosidase, was obtained from Eurogentec (Seraing, Belgium). The plasmid encoding a dominant negative form of IKK-β was a generous gift from Dr. J. Schmid (University of Vienna, Vienna, Austria). Plasmids encoding T7-epitope tagged COP/Pseudo-ICE and ICEBERG have been described previously (Druilhe et al., 2001) and were kindly provided by Dr. E. S. Alnemri (Thomas Jefferson University, Philadelphia, Pa., USA).

The entire open reading frame of INCA was amplified by PCR using complementary PCR adaptor primers spanning the initiation and stop codons of INCA. Subsequently, the PCR products were cloned in frame with the E-epitope or Flag-epitope tag of the expression vectors pCAGGS-E or pCAGGS-Flag vector, respectively. The PCR-generated cDNAs encoding the ORF of human RIP2, COP/Pseudo-ICE, ICEBERG and human caspase-2 CARD were all cloned in frame with the E-epitope tag of the pCAGGS-E vector. The enzymatically inactive human procaspase-1 C285A mutant was made by site-directed mutagenesis PCR and cloned in frame with the E-epitope tag of the pCAGGS-E vector. All the PCR products described above were checked by sequencing to ensure that no errors had been introduced by PCR.

Transfection, Co-Immunoprecipitation and Immunoblotting Assay

293T is a human embryonal kidney carcinoma cell line. 293T cells were routinely transfected using the calcium phosphate precipitation method (O'Mahoney and Adams, 1994). Cells were seeded the day before transfection at 2×10⁵ cells/6-well. Cells were transfected for 4 h, washed and incubated for another 24 h before lysates were prepared by harvesting the cells and lysing them in ice-cold NP-40 lysis buffer (10 mM HEPES pH 7.4, 142.5 mM KCl, 0.2% NP-40, 5 mM EGTA), supplemented with 1 mM DTT, 12.5 mM β-glycerophosphate, 1 mM Na₃VO₄, 1 mM PMSF, and 1× protease inhibitor mix (Roche, Basel, Switzerland). Cell lysates (0.5 ml) were clarified by centrifugation at 14,000 g for 5 minutes, and subjected to immunoprecipitation using anti Flag antibodies (Sigma, St. Louis, Mo., USA) in combination with 15 μl Protein G-Sepharose. Immune-complexes were fractionated by sodium dodecyl sulfate-polyacrylamide gel electroforesis (SDS-PAGE) and transferred to nitrocellulose membranes. The blots were subsequently incubated with anti-E antibodies (Amersham Biosciences, Freiburg, Germany), followed by horseradish peroxidase-conjugated secondary antibodies, and detection by an enhanced chemiluminescence (ECL) method. Alternatively, lysates were analyzed directly by immunoblotting after normalization for total protein content.

Retroviral Infection of THP-1 Cells

The monocytic cell line THP1 was cultured at 37° C. under 6% CO₂ in RPMI 1640 supplemented with 10% FCS, L-glutamine (2 mM), penicillin (100 units/ml), streptomycin sulfate (100 μM), sodium pyruvate (1 mM), β-mercaptoethanol (10⁻⁵ M). The amphotropic packaging cell line Phoenix (G.P. Nolan's laboratory, Stanford University Medical Center, Stanford, Calif., USA) was transfected with pFBneo, pFBneo-INCA, pFBneo-ICEBERG, pFBneo-COP vectors using the calcium phosphate/chloroquine method. The next day cultures were refreshed. Culture supernatants containing retroviral particles were collected 24 hours later and filtered through a 0.45 μm membrane. 1 ml of viral supernatant was incubated with 10 μl DOTAP (Roche) for 10 min on ice. THP1 cells (10⁶ cells/well) were centrifuged in the presence of 1 ml of retrovirus enriched with DOTAP in a 6-well plate for 45 min at 1200 r.p.m. at 24° C. Plates were placed back in a CO₂ incubator at 37° C., 6 hours later fresh medium was added, and the cells were kept in culture for 18 hours. THP1 cells were subjected to a total of three cycles of infection followed by 1 week of culture. Cells were then selected using 1.5 mg/ml neomycin (Life Technologies). After 4 weeks of selection, the cultures were expanded and expression of INCA, ICEBERG and COP were verified by Western blotting.

Mature IL-1β Bio-Assay

Biologically active IL-1β was determined using growth factor-dependent D10(N4)M cells (Hopkins and Humphreys, 1989). Cells were maintained in RPMI 1640 medium supplemented with 10% FCS, 2 mM L-glutamine, 100 IU/ml penicillin G, 100 μg/ml streptomycin, 1 mM sodium pyruvate, 5 mM β-mercaptoethanol and 10% supernatant of phorbol ester-stimulated EL-4 cells as a source of IL-2, and 10% supernatant of phorbol ester-stimulated P388D1 cells as a source of IL-1. The day before the assay, D10(N4)M cells were washed and transferred to fresh media containing 10% EL-4 supernatant. The next day, cells were washed again and added to serial dilutions of IL-1β-containing samples (10⁴ cells/96-well), followed by incubation for 24 h at 37° C. in a CO₂ incubator. Proliferation was quantified by [³H]thymidine incorporation (0.5 μCi/well) for the last 6 h. Cells were harvested and incorporated [³H]thymidine was determined in a microplate scintillation counter (Packard Instrument Co., CT, USA). Samples were quantified according to a standard preparation of IL-1β with a specific biological activity of 10⁹ IU/mg (obtained from the National Institute for Biological Standards and Control, Potters Bar, UK).

Quantification of NF-κB Activity

293T cells were transfected with the indicated expression vectors in combination with 100 ng NF-κB-luciferase and pUT651-β-galactosidase reporter plasmids. In some experiments, cells were treated for 6 h with 500 IU/ml of TNF-α prior to harvesting. Forty-eight hours after transfection the cells were collected, washed in phosphate buffered saline and lysed in Tris phosphate (25 mM, pH 7.8), 2 mM DTT, 2 mM CDTA, 10% glycerol and 1% Triton-X100. After addition of 50 μl substrate buffer (658 μM luciferin, 378 mM co-enzyme A and 742 μM ATP) to 20 μl of cell lysates, NF-κB activity was assayed in a TopCount NXT microplate scintillation reader (Packard Instrument Co, Meriden, Conn., USA). To normalize transfection efficiency, cell lysates were also subjected to β-galactosidase colorimetric assay. In brief, 20 μl of cell lysate were incubated for 5 min at room temperature with 200 μl of a solution containing 0.9 mg/ml o-nitrophenyl-β-D-galactopyranoside, 1 mM MgCl₂, 45 mM β-mercaptoethanol and 100 mM sodium phosphate, pH 7.5. The optical density was read at a wavelength of 595 nm. Results are expressed as relative luciferase units per second/optical density for β-galactosidase activity. The data represent the average ±S.E. of at least three independent experiments.

### Example 1

### Identification of INCA

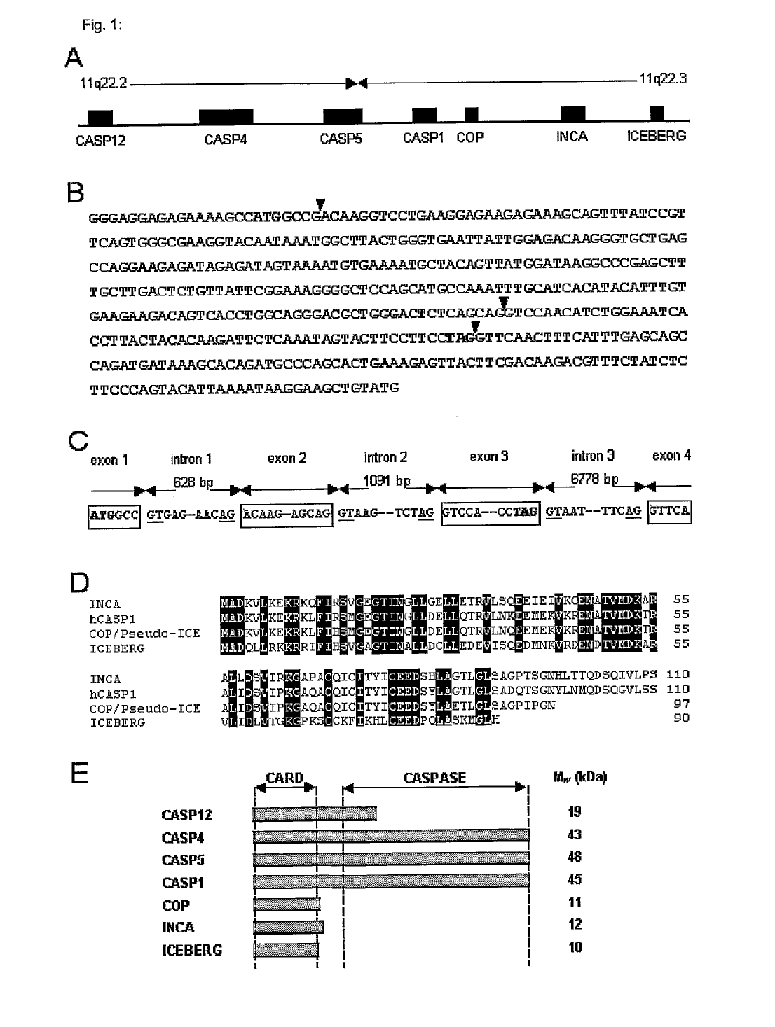

To identify new CARD-containing proteins, we searched the GenBank™ High Throughput Genomic Sequence (HTGS) database for sequences that share significant homology to the prodomain of human caspase-1 (residues 1-100). Using this approach, four different genomic clones (GenBank accession numbers AP002787, AC027011, AP001024, AC021452) containing a new CARD-containing gene were found. The identified gene, which we named INCA (Inhibitory CARD), maps to human chromosome 11q22. Interestingly, the genes coding for caspase-1, the related CARD-proteins ICEBERG and COP/Pseudo-ICE and caspases-4, -5 and -12 all reside on this locus. According to the public database of Human Genome Browser (http://genome.ucsc.edu/), the order of these genes from centromere to telomere is caspase-12, caspase-4, caspase-5, caspase-1, COP/Pseudo-ICE, INCA and ICEBERG (FIG. 1A). Since INCA shares high sequence homology with the genes encoding caspase-1, COP/Pseudo-ICE and ICEBERG, it is likely that the INCA gene arose from a duplication of one of these homologous genes.

To deduce the putative cDNA sequence of INCA, we aligned its genomic sequence with the cDNA sequences of procaspase-1, COP/Pseudo-ICE and ICEBERG to predict potential exons and intron/exon boundaries. The results of this approach largely matched those obtained using de novo gene prediction methods such as GenScan and GeneMark.hmm. The predicted INCA cDNA sequence is composed of four exons (FIG. 1B) with all intron/exon boundaries conform to the consensus GT/AG rule (FIG. 1C). The open reading frame spans from the first to the third exon, which encodes an in frame stop codon (FIG. 1B). Only the first two amino acids are encoded in the first exon and the last 18 amino acids are encoded by exon 3. Therefore, exon 2 encodes most of the ORF, including the CARD domain. Exon 4 is not coding for amino acids because it resides downstream of the in frame stop codon at the end of exon 3 and thus functions as a 3′-untranslated region (FIG. 1B). The deduced amino acid sequence of INCA shares 81% sequence identity with the CARD-domain of procaspase-1 (FIG. 1D). These data show that the INCA gene probably encodes a protein of 110 amino acids (FIG. 1D), which essentially consists of a CARD domain (residues 1-91). INCA is therefore comparable to the related CARD-only proteins COP/Pseudo-ICE, ICEBERG and human caspase-12 (FIG. 1E) (Fischer et al., 2002; Lamkanfi et al., 2004b; Saleh et al., 2004), all encoded by genes residing on the same chromosomal locus.

### Example 2

### Tissue Expression of INCA

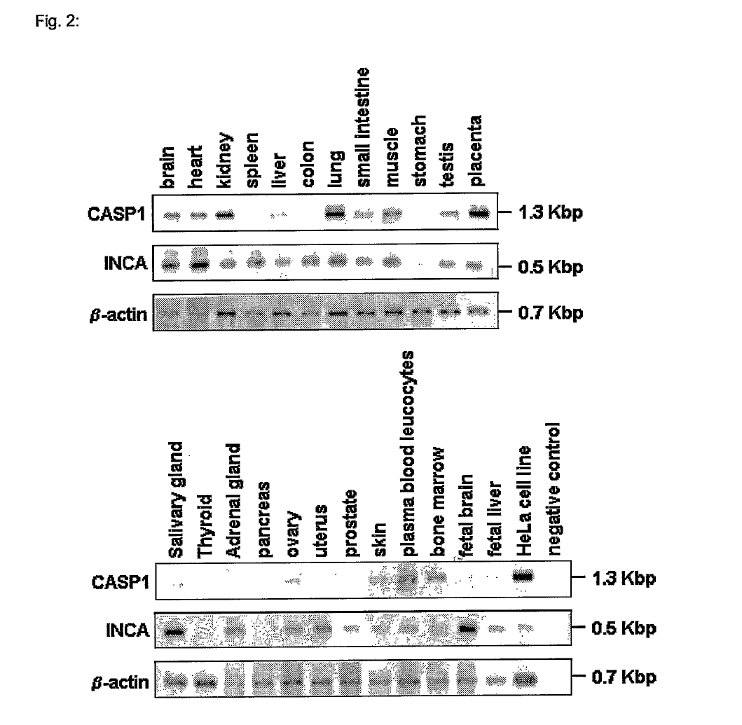

Using the nucleotide or amino acid sequences of INCA as a query for BLAST searches of the GenBank™ database, no EST sequences corresponding to INCA could be identified. To experimentally confirm the existence and to study the tissue distribution of the predicted INCA mRNA, we performed RT-PCR analysis using INCA-specific primers on a cDNA panel derived from several normal human tissues and from the human cervix carcinoma cell line HeLa. Parallel PCR analysis of procaspase-1 and β-actin mRNA served as a reference (FIG. 2). INCA-specific primers amplified a PCR product of about 470 bp in length from several tissues, with highest expression levels detected in brain, heart, spleen, lung and salivary gland (FIG. 2). Subsequent DNA sequencing of this PCR product confirmed the predicted INCA cDNA sequence. INCA was absent or expressed at low levels in various other tissues, including stomach, thyroid, pancreas, prostate and skin, as well as in HeLa cells (FIG. 2). In general, INCA is expressed in most tissues where procaspase-1 is present. However, in a number of tissues, such as salivary gland, INCA is expressed in the absence of procaspase-1 (FIG. 2). This suggests that differential regulation mechanisms at the transcriptional or post-transcriptional level control these homologous genes.

### Example 3

### INCA is Upregulated by IFN-γ

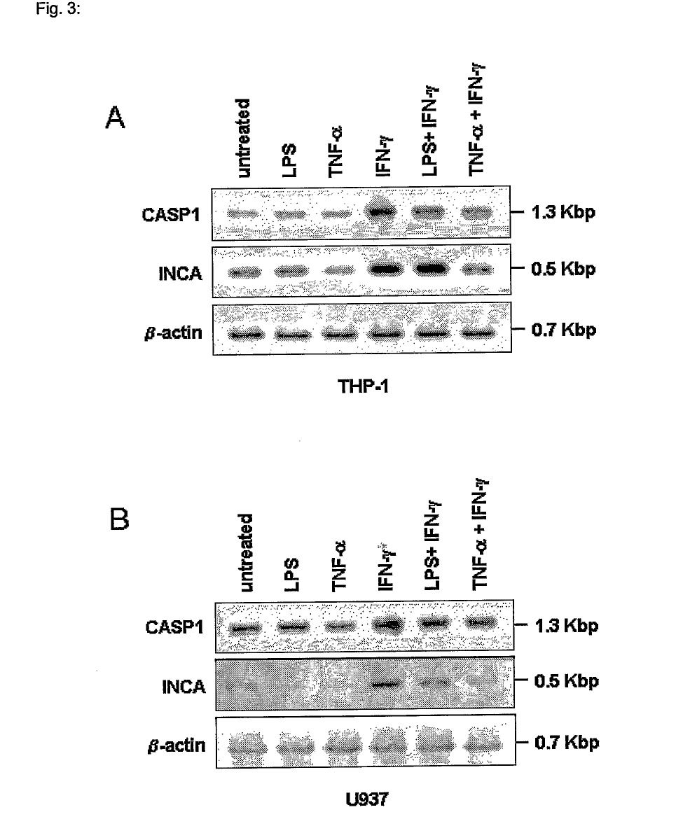

To analyze the existence of possible shared regulation mechanisms between procaspase-1 and INCA, we compared the modulation of INCA and procaspase-1 mRNA levels in response to various pro-inflammatory stimuli. Caspase-1 mRNA levels are known to be upregulated when cells are stimulated with IFN-γ, but remain unchanged following LPS- or TNF-stimulation (Chin et al., 1997; Kalai et al., 2003; Lin et al., 2000; Tamura et al., 1996). Following stimulation of the monocytic cell lines U937 and THP-1, we analyzed the induction profiles of INCA and caspase-1 by RT-PCR using INCA- and procaspase-1-specific primers, respectively (FIG. 3). Procaspase-1 mRNA levels were indeed strongly induced by IFN-γ in both cell lines, while remaining largely unchanged in LPS- and TNF-α stimulated cells (FIG. 3). These results confirm and extend published data on the induction profile of caspase-1 (Chin et al., 1997; Kalai et al., 2003; Lin et al., 2000; Tamura et al., 1996). Comparable to procaspase-1, treatment of U937 or THP-1 cells with IFN-γ leads to a significant upregulation of INCA levels, while remaining unchanged in LPS-stimulated cells (FIG. 3). These results indicate that procaspase-1 and INCA mRNA levels are both specifically upregulated by IFN-γ. Interestingly, we noticed that TNF-α is capable of down regulating the IFN-γ-induced upregulation of procaspase-1 and INCA in both THP-1 and U937 cells (FIG. 3). In both cell lines, we observed that this IFN-γ-modulating effect of TNF-α is more pronounced for INCA than for procaspase-1 (FIG. 3). All together, these results suggest that INCA and procaspase-1 mRNA levels are modulated in similar ways, though the strength of the response to a certain stimulus can vary.

### Example 4

### Identification of INCA-Interacting Proteins

The prodomain of procaspase-1 is required for dimerization and activation of the zymogen (Van Criekinge et al., 1996). Because INCA shares a high degree of amino acid sequence identity with the prodomain of procaspase-1 (FIG. 1), we tested the possibility that INCA interacts with procaspase-1 in co-immunoprecipitation assays. Interactions with several other CARD-containing proteins were also tested, including INCA itself, the related CARD-only proteins ICEBERG and COP/Pseudo-ICE. Because it has been demonstrated that procaspase-1 and COP/Pseudo-ICE interact with the CARD-containing kinase RIP2 to induce NF-κB activation (Druilhe et al., 2001; Lamkanfi et al., 2004a), we also tested the interaction of INCA with this kinase. The unrelated CARD domain of procaspase-2 was used as a negative control for the co-immunoprecipitation assays. For these experiments, 293T cells were transiently transfected with expression plasmids encoding Flag-tagged INCA in combination with various other expression plasmids producing E-tagged CARD-containing proteins. Immunoprecipitations were then performed with anti-Flag antibody, and the resulting immunocomplexes were analyzed by SDS-PAGE and immunoblotting using anti-E antibody. Aliquots of the lysates were also analyzed directly by immunoblotting to verify the production of each protein.

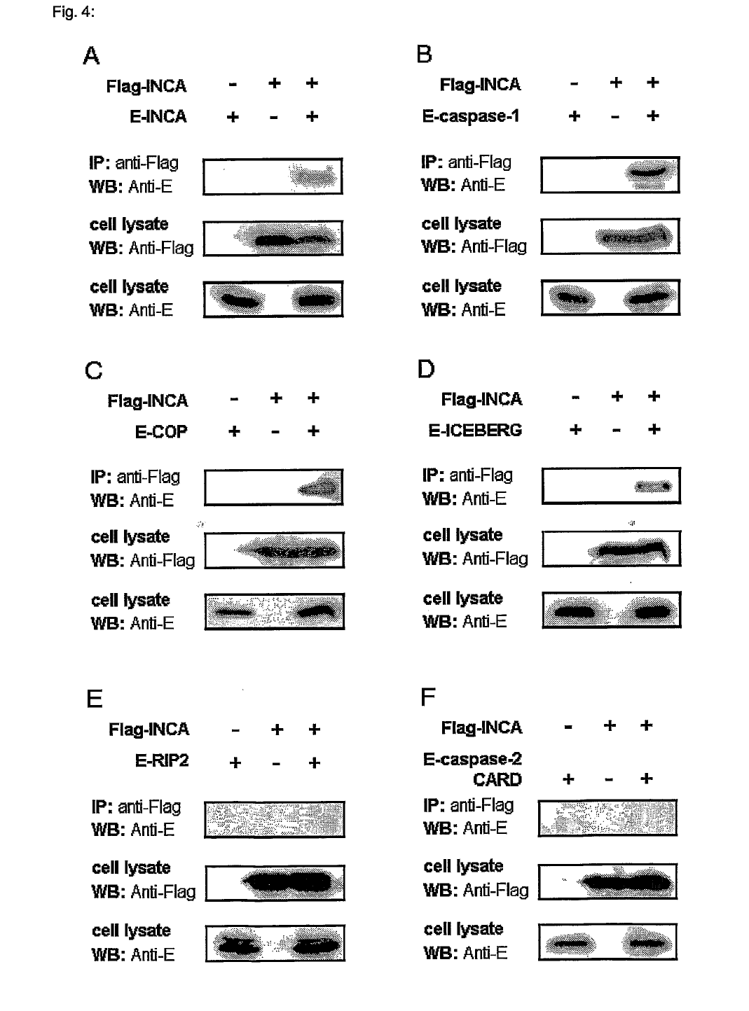

E-INCA co-immunoprecipitated with Flag-INCA, indicating that this protein can self-associate (FIG. 4A). In addition, E-procaspase-1 co-immunoprecipitated with Flag-INCA (FIG. 4B), suggesting that INCA can bind to the prodomain of procaspase-1. Note that the active site cysteine of procaspase-1 was mutated to alanine for these co-immunoprecipitation experiments to avoid induction of apoptosis. Finally, the CARD-only proteins COP/Pseudo-ICE and ICEBERG also co-immunoprecipitated with Flag-INCA (FIG. 4C en D), indicating that these highly related CARD domains that bind to the prodomain of procaspase-1, can also cross-associate with the similar CARD domain present in INCA. In contrast with what was reported for COP/Pseudo-ICE (Druilhe et al., 2001; Lee et al., 2001), but similar to ICEBERG (Druilhe et al., 2001; Humke et al., 2000), E-RIP2 did not co-immunoprecipitate with Flag-INCA (FIG. 4E). This suggests that COP/Pseudo-ICE contains a RIP2-binding interface at the surface of its CARD domain, which is not present in the more distantly related CARD domains of INCA and ICEBERG. E-procaspase-2 CARD also did not co-immunoprecipitate with Flag-INCA (FIG. 4F), thus demonstrating the specificity of these results.

### Example 5

### Comparative Analysis of the Capacity of CARD-Only Proteins to Modulate NF-κB

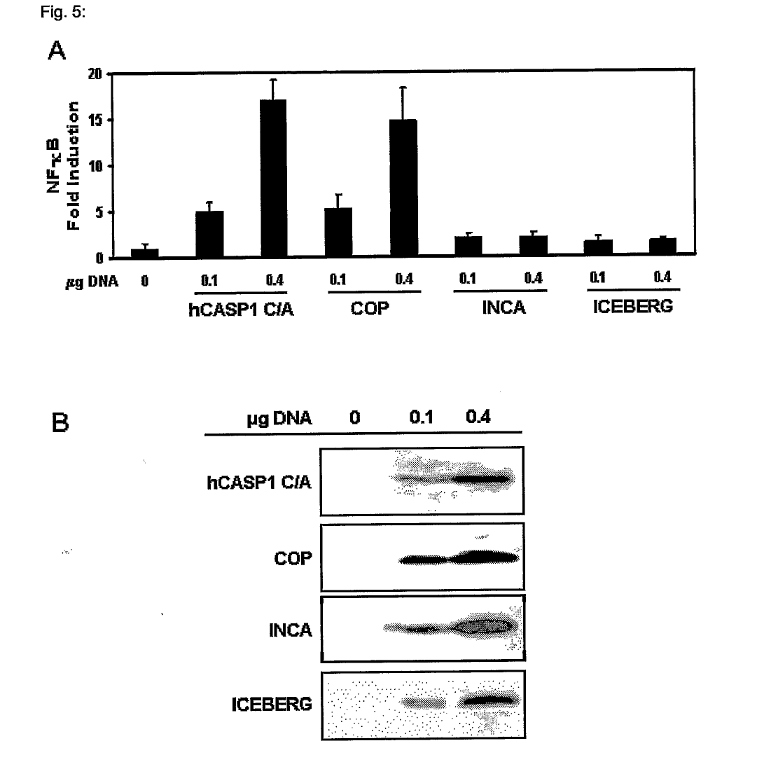

We have recently demonstrated that caspase-1 CARD also potently activates the transcription factor NF-κB in a RIP2-dependent manner (Lamkanfi et al., 2004a). COP/Pseudo-ICE also interacts with RIP2 and induces NF-κB activation upon overexpression in 293T cells (Druilhe et al., 2001). However, ICEBERG does not interact with RIP2 and is unable to activate NF-κB (Druilhe et al., 2001). ICEBERG shares 53% sequence identity with caspase-1 CARD while INCA and COP/Pseudo-ICE share 81% and 93% sequence identity with the prodomain of caspase-1, respectively. Thus, INCA is intermediate between COP/Pseudo-ICE and ICEBERG. Therefore, we tested whether INCA is capable of inducing NF-κB activity. 293T cells were co-transfected with an NF-κB-driven luciferase reporter plasmid and plasmids encoding either empty vector, enzymatically inactive caspase-1 C285A, COP/Pseudo-ICE, INCA or ICEBERG. As expected, both procaspase-1 C285A and COP/Pseudo-ICE potently induced NF-κB activity (FIG. 5). Like ICEBERG, INCA was completely incapable of activating NF-κB (FIG. 5), even when very high plasmid concentrations were used. This result correlates with the observation that INCA does not interact with the NF-κB-activating kinase RIP2 (FIG. 4E). In conclusion, unlike procaspase-1 CARD and COP/Pseudo-ICE, INCA and ICEBERG are unable to induce NF-κB activation.

### Example 6

### INCA does not Inhibit NF-κB Activation Induced by TNF, Caspase-1, COP/Pseudo-ICE or RIP2

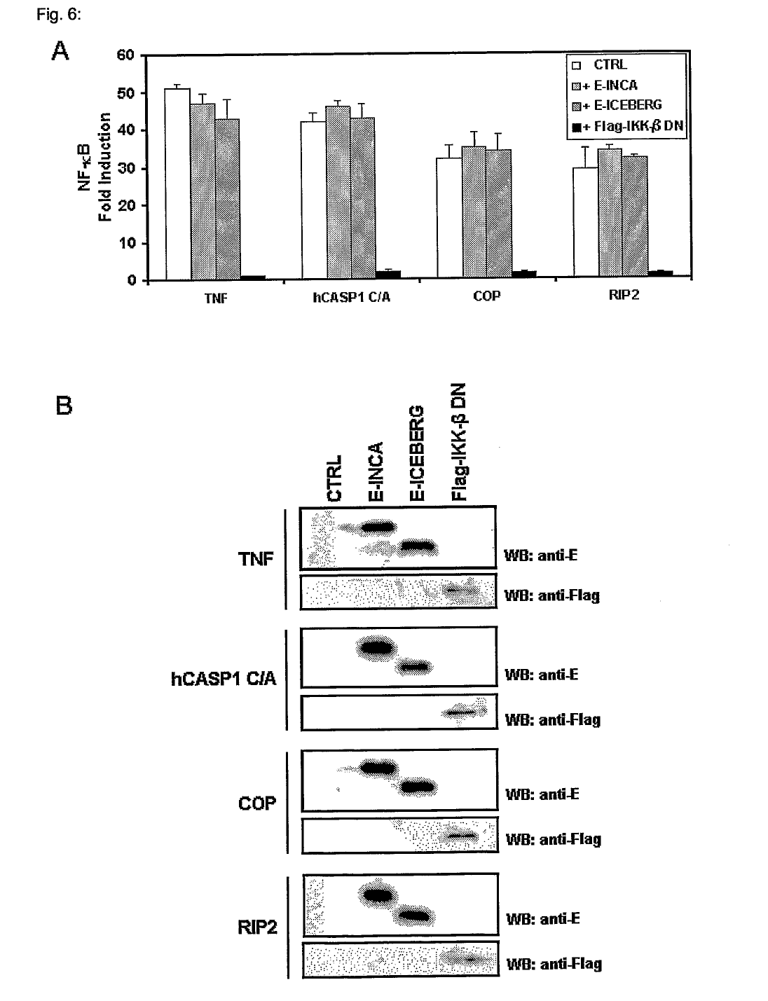

Several recently cloned CARD-containing proteins have been shown to inhibit rather than to induce NF-κB activity (Razmara et al., 2002; Stehlik et al., 2003). For example, CARD-8 is known to inhibit both RIP2- and TNF-induced NF-κB activation (Razmara et al., 2002). As both INCA and ICEBERG are unable to induce NF-κB activation (FIG. 5), we investigated whether they can inhibit NF-κB activity induced by TNF, caspase-1, COP/Pseudo-ICE or RIP2. Since most NF-κB signaling pathways converge at the IKK-complex, we used a dominant negative form of IKK-β (IKK-β DN) as a positive control for inhibition. As expected, IKK-β DN completely abolished NF-κB activity from the four activating molecules (FIG. 6A). However, INCA and ICEBERG did not significantly affect TNF-, caspase-1-, COP/Pseudo-ICE- or RIP2-induced NF-κB activation (FIG. 6A), though Western blotting analysis confirmed the appropriate expression of both CARD-proteins (FIG. 6B). All together, these data suggest that INCA and ICEBERG do not function as endogenous modulators of the studied NF-κB signaling pathways.

### Example 7

### INCA Inhibits the Release of IL-1β from THP-1 cells

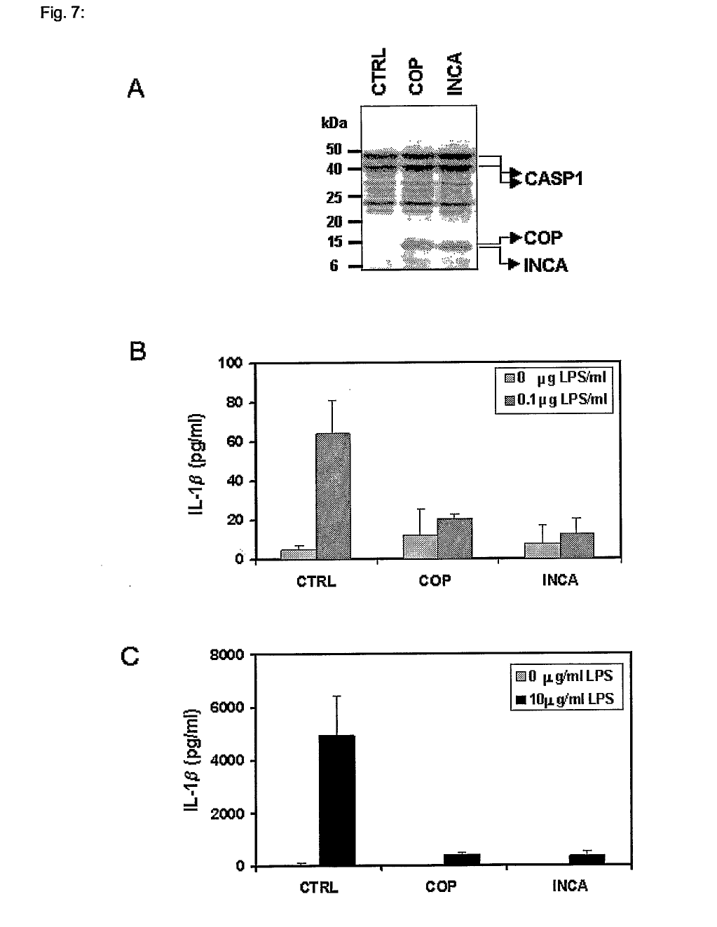

THP-1 monocytes release IL-1β in response to inflammatory stimuli such as LPS. The processing of pro-IL-1β to the 17.5 kDa mature form and its release are well-known consequences of caspase-1 activation (Kuida et al., 1995; Li et al., 1995). The INCA-related CARD-only proteins ICEBERG and COP/Pseudo-ICE have been shown to significantly blunt IL-1β maturation following LPS-stimulation of THP-1 cells (Druilhe et al., 2001; Humke et al., 2000). To test whether INCA resembles ICEBERG and COP/Pseudo-ICE in this feature, we generated stable transfectants of THP-1 cells expressing Flag-tagged INCA under the control of a retroviral promoter. Stable transfectants of THP-1 cells expressing Flag-tagged COP/Pseudo-ICE were used as a positive control in this experiment. The stable transfectants expressed INCA and COP/Pseudo-ICE at levels comparable to the constitutive expression of endogenous procaspase-1 in THP-1 cells (FIG. 7A). As expected, neither COP/Pseudo-ICE nor INCA-expressing cells released mature IL-1β in unstimulated cells (FIGS. 7B and C). In accordance with published results (Druilhe et al., 2001), COP/Pseudo-ICE-expressing cells produced significantly lower amounts of mature IL-1β in response to both low and high concentrations of LPS, when compared to mock-transfected control cells (FIGS. 7B and C). INCA was as effective as COP/Pseudo-ICE in inhibiting IL-1β generation at both doses of LPS used in this experiment (FIGS. 7B and C). Taken together, our results show that INCA significantly reduces the release of mature IL-1β in monocytic THP-1 cells and suggest that the binding of INCA to procaspase-1 prevents the CARD-mediated activation of the enzyme (Martinon et al., 2002; Van Criekinge et al., 1996).

### REFERENCES

Cerretti, D. P., C. J. Koziosky, B. Mosley, N. Nelson, K. Van Ness, T. A. Greenstreet, C. J. March, S. R. Kronheim, T. Druck, L. A. Cannizzaro, and et al. 1992. Molecular cloning of the interleukin-1 beta converting enzyme. Science. 256:97-100.
Chin, Y. E., Kitagawa, M., Kuida, K., Flavell, R. A., and Fu, X. Y. (1997). Activation of the STAT signaling pathway can cause expression of caspase 1 and apoptosis. Mol Cell Biol 17, 5328-5337.
Dinarello, C. A., and Wolff, S. M. (1993). The role of interleukin-1 in disease. N Engl J Med 328, 106-113.
Druilhe, A., Srinivasula, S. M., Razmara, M., Ahmad, M., and Alnemri, E. S. (2001). Regulation of IL-1 beta generation by Pseudo-ICE and ICEBERG, two dominant negative caspase recruitment domain proteins. Cell Death Differ 8, 649-657.
Fischer, H., Koenig, U., Eckhart, L., and Tschachler, E. (2002). Human caspase 12 has acquired deleterious mutations. Biochem Biophys Res Commun 293, 722-726.
Ghayur, T., S. Banerjee, M. Hugunin, D. Butler, L. Herzog, A. Carter, L. Quintal, L. Sekut, R. Talanian, M. Paskind, W. Wong, R. Kamen, D. Tracey, and H. Allen. 1997. Caspase-1 processes IFN-gamma-inducing factor and regulates LPS-induced IFN-gamma production. Nature. 386:619-23.
Gu, Y., K. Kuida, H. Tsutsui, G. Ku, K. Hsiao, M. A. Fleming, N. Hayashi, K. Higashino, H. Okamura, K. Nakanishi, M. Kurimoto, T. Tanimoto, R. A. Flavell, V. Sato, M. W. Harding, D. J. Livingston, and M. S. Su. 1997. Activation of interferon-gamma inducing factor mediated by interleukin-1 beta converting enzyme. Science. 275:206-9.
Hopkins, S. J., and Humphreys, M. (1989). Simple, sensitive and specific bioassay of interleukin-1. J Immunol Methods 120, 271-276.
Humke, E. W., Shriver, S. K., Starovasnik, M. A., Fairbrother, W. J., and Dixit, V. M. (2000). ICEBERG: a novel inhibitor of interleukin-1 beta generation. Cell 103, 99-111.
Kalai, M., Lamkanfi, M., Denecker, G., Boogmans, M., Lippens, S., Meeus, A., Declercq, W., and Vandenabeele, P. (2003). Regulation of the expression and processing of caspase-12. J Cell Biol 162, 457-467.
Kuida, K., Lippke, J. A., Ku, G., Harding, M. W., Livingston, D. J., Su, M. S., and Flavell, R. A. (1995). Altered cytokine export and apoptosis in mice deficient in interleukin-1 beta converting enzyme. Science 267, 2000-2003.
Lamkanfi, M., Kalai, M., Saelens, X., Declercq, W., and Vandenabeele, P. (2004a). Caspase-1 activates NF-kappa B independent of its enzymatic activity. J Biol. Chem.
Lamkanfi, M., Kalai, M., and Vandenabeele, P. (2004b). Caspase-12: an overview. Cell Death Differ 11, 365-368.
Lee, S. H., Stehlik, C., and Reed, J. C. (2001). Cop, a caspase recruitment domain-containing protein and inhibitor of caspase-1 activation processing. J Biol Chem 276, 34495-34500.
Li, P., Allen, H., Banerjee, S., Franklin, S., Herzog, L., Johnston, C., McDowell, J., Paskind, M., Rodman, L., Salfeld, J., and et al. (1995). Mice deficient in IL-1 beta-converting enzyme are defective in production of mature IL-1 beta and resistant to endotoxic shock. Cell 80, 401-411.
Lin, X. Y., Choi, M. S., and Porter, A. G. (2000). Expression analysis of the human caspase-1 subfamily reveals specific regulation of the CASP5 gene by lipopolysaccharide and interferon-gamma. J Biol Chem 275, 39920-39926.
Martinon, F., Burns, K., and Tschopp, J. (2002). The inflammasome: a molecular platform triggering activation of inflammatory caspases and processing of proIL-beta. Mol Cell 10, 417-426.
O'Mahoney, J. V., and Adams, T. E. (1994). Optimization of experimental variables influencing reporter gene expression in hepatoma cells following calcium phosphate transfection. DNA Cell Biol 13, 1227-1232.
Razmara, M., Srinivasula, S. M., Wang, L., Poyet, J. L., Geddes, B. J., DiStefano, P. S., Bertin, J., and Alnemri, E. S. (2002). CARD-8 protein, a new CARD family member that regulates caspase-1 activation and apoptosis. J Biol Chem 277, 13952-13958.
Saleh, M., Vaillancourt, J. P., Graham, R. K., Huyck, M., Srinivasula, S. M., Alnemri, E. S., Steinberg, M. H., Nolan, V., Baldwin, C. T., Hotchkiss, R. S., et al. (2004). Differential modulation of endotoxin responsiveness by human caspase-12 polymorphisms. Nature 429, 75-79.
Stehlik, C., Hayashi, H., Pio, F., Godzik, A., and Reed, J. C. (2003). CARD6 is a modulator of NF-kappa B activation by Nod1- and Cardiak-mediated pathways. J Biol Chem 278, 31941-31949.
Tamura, T., Ueda, S., Yoshida, M., Matsuzaki, M., Mohri, H., and Okubo, T. (1996). Interferon-gamma induces Ice gene expression and enhances cellular susceptibility to apoptosis in the U937 leukemia cell line. Biochem Biophys Res Commun 229, 21-26.
Van Criekinge, W., Beyaert, R., Van de Craen, M., Vandenabeele, P., Schotte, P., De Valck, D., and Fiers, W. (1996). Functional characterization of the prodomain of interleukin-1beta-converting enzyme. J Biol Chem 271, 27245-27248.

1. A nucleic acid encoding a CARD only protein, said nucleic acid comprising SEQ ID NO:3.

2. A method of inhibiting caspase-1 activity, said method comprising:
using a card-only protein comprising SEQ ID NO:2, or a functional fragment thereof to inhibit caspase-1 activity.

3. A method of inhibiting pro-interleukin-1β maturation, said method comprising:
using a card-only protein comprising SEQ ID NO:2, or a functional fragment thereof to inhibit pro-interleukin-1β maturation.

4. The method according to claim 2, wherein said inhibition is not accompanied by NF-κB induction.

5. The method according to claim 2, wherein said inhibition is not accompanied by apoptosis.

6. The method according to claim 3, wherein said inhibition is not accompanied by NF-κB induction.

7. The method according to claim 3, wherein said inhibition is not accompanied by apoptosis.

8. A method of inhibiting inflammation in a subject suffering therefrom, said method comprising:
administering to the subject a peptide comprising SEQ ID NO:2, or a fragment thereof having pro-interleukin-1β maturation inhibitory activity in the subject.

9. The method according to claim 8, wherein NF-κB is not induced in the subject.

10. The method according to claim 8, wherein apoptosis in not induced in the subject.


SEQUENCE LISTING (10 sequences)

SEQ ID NO: 1 - DNA, 466 bp, Mus musculus
  CDS (18)..(350)
gggaggagagaaaagccatggccgacaaggtcctgaaggagaagagaaagcagtttatccgttcagtgggcgaaggtacaataaatggcttactgggtgaattattggagacaagggtgctgagccaggaagagatagagatagtaaaatgtgaaaatgctacagttatggataaggcccgagctttgcttgactctgttattcggaaaggggctccagcatgccaaatttgcatcacatacatttgtgaagaagacagtcacctggcagggacgctgggactctcagcaggtccaacatctggaaatcaccttactacacaagattctcaaatagtacttccttcctaggttcaactttcatttgagcagccagatgataaagcacagatgcccagcactgaaagagttacttcgacaagacgtttctatctcttcccagtacattaaaataaggaagctgtatg

SEQ ID NO: 2 - protein, 110 aa, Mus musculus
MADKVLKEKRKQFIRSVGEGTINGLLGELLETRVLSQEEIEIVKCENATVMDKARALLDSVIRKGAPACQICITYICEEDSHLAGTLGLSAGPTSGNHLTTQDSQIVLPS

SEQ ID NO: 3 - DNA, 22440 bp, Mus musculus
  exon (6087)..(6110)
  exon (6739)..(7005)
  exon (8097)..(8155)
  exon (14934)..(15049)
taattgactcacagccggctgtgtgggtgatcagagttttgttattacttgaatcagtctccccgagcattcagaaatcagaatgtttaaagataatttggcaggtaagggctagggaagtagggagtgctgattagttaggtgggagatggaatcatagggtgtcaaagtgaggttttcttgctgtcttctcttcctgggtgctatggcagcctggttgagttagattatgggtcggggtggtgtcagctgatccactgagtgcagtgtctgaaaaatatcttaagtgctgatcttaggtttcacaatagtgatattatccccagagcatgttggggacgttcagactctaggagccagaggttgcatgacccttaaattgtaatttttaatcttgtagctaatttgttagtcctgcaaaggcagactggaccccaggcaagaaggggtctttttgggaaaggactgtaatcaattttgtttcagagtcagaccatgaactgaattccttcccaaagttagttcagcctatgcccatgaatgaagaaggacagcttaagtgttagaagcaagatggagttggttaggtctgatttctttcactgtcataatttcctcagttgcaatattataaaggcggtttcaatcccttcctttgggttttataacagcttaatcttaagatgtaggctatgaagatgggaaaaggctgttgatcacactggcttccatctgctgacaggggacatagtggaaattggagtgaaccccaaggtgagaagactggaactgctttgcaactgccagaccatacttatgcaggcctggctgggcttccaatgcttacatggcaaaaacattagtattgtccttgtaggtttcctacagtattcaagagaacagcctcctgtaaggtataaaacaaatcctaggataaggagtaccattctcaattttaaaaggaaagattctaaaaagaattttaaaaggaaagatttgaaagcactagtttagagacttctaatccacaaatattttgggatttattccaaattgcagaaaaaaaaaacctcaaggacagctaacaacaggtgtattatagattttctttcaaagcataatttttctctttccagactcatttttattaagaacaaattatgatagaaataatttgtttacaaaaataagctttagtcttactgtacttgacctgattatttgcataaggtgcaacagataattatttttcagttaggctgttttttgtttaaattggctttgatggaactctgttccatgagcaatctcaggtaagacttttcaagagccgagtccagccatgcgttcgtacactcaaatacctatgagtggggtaaattcctctcctctcaaggtcccaagataaattggagctcctgaacctgttagacagtgatattcttcacttaccagagatcaagaaacttgtacaaggactctgtgtagacaaggtatgaagccagattccccaagggatttcattgtttctatgagtcaacttcgattctttaaaagaagcacagcattccagtcaaagccttgataaaacaaccaatttctccgattgtgtcctgttacaaaaaaaaaaaaaaaaaaacaaaaaatcttatgcacttatgcaattaactatgctcccataaattgtgaatacttacaaatagcttccaaattctggagaaatcagatagagagaaacaaagatggttcaaaattttttcaccagagcatattttactcacttgttaaaagttgcaaatagctctaaagaaataagttatcttgactctgaaaacaaaaggattagtgatatttaacatataaggtcttcatgagagtcctagaagtttcattttttcctccattccaatagcacaatttttaaagttatctgacacctgcattcaaagtcctatatctgattataaactgccatttgaaaaggaccaaagcaagacaaaatatctgttgatgacaaaagcctatagacactattaaagttataattgactaggaatttggcttacttctgtggcatcccacaattttacataacaatgataattactaataatgtacactaaataatatcagaattacagaagtttatataaatacatggacagaaaactccgggcttgtaagatttttcatttgccagctttaaattggattactagcttcagggtggagcccttggaggaacagggtcaggaaagcatgcatttttttctgagctaaataagcagccacagctgaagggaaagacagatctccagaattaagggtgtcattttatactggatgcttgatccccaaaaggaggaaaatactaccagagaagacagtgtggtgcttctaccctgcattttgttgcaaggcaacctaaagccaatcagcccattttgaaatcagcccatcccccatggcaatctcatctctcagtcaagggtaaggatgtttccatatcttccaggtgtccaagtgcatgtttctctaatccaagtgtgcaaagagccaagtatgcctccaaaaatactcttggccatccattaaatatatttcctacctagttattacataccaaagctctctcataatgcaaagtaatttctgatccccccaaaactcaaactgtcagataacacaatgcaaaacagaatagagcctttgattttgatagggatctatctactgttaattcctggggtttcgggaagaaaacagagcggtttctttttgtttgctttttgttgttgttttttttccaaaacgaggtctatggtgcctcctctgtttttcccaaggagtcgcaggctaccagaagttattttagggcctctcatgtgtgcattaaaagtggcaagacaaaaaatggaaaaaaataattcagttgactaagaagaaaaaaactcccccctcagataagtagtgattgaatttctatcaatatacgttagtttttttctaatttagaactttatataattggaatcccagggtatgtgttccctcctatatggcttcattcactcataatgaatgcagcagttttttcattcatattattacatatcttagtaatatttcccttttatgactgaacagcatttcagtgtatgtatatagcacaaattatttacttatgctcctgaattggttattgagttgtttccagtttttgacatctccatttttgaggaacagtgggcccttgcaatctagttgctcaatgtttagcttcccaggacatgagactcaaggcttatggttctacatccccagatatatggtctagtcatgatttctgtgtcctggatgccgctttggcatgcttcaactcagtggttcctggctctatctggggaagaaaccttcacgtgaaagttaacagaaaaaataaattaatcagtaaataaatttaaaagcaaatgattggaagagaagtaatttacaggcaatttctgctttctttcatgctacaaatcctcccagtggcaaggagcacaagattgtgctgcaaatggattaagtcattgccaaagtaaagataaaactttgcctgattttctcaccccagaaagtttttcatgcccattgtactcaatttcctgccacttcctcctaagtagtgattaaatttttaccactatacattagttatttctaatataaaccttatttaattggaatcacaggatatatgttcctttgtatatggcttctttcactcatcatgaaatcataatgttgtcatccatgttactgcataaattagtatttttttcttttgtgactgaatagtatttcagtgtatgtgtatagcacaaattgtttacttatgctcctgaattggatattgaattgttgccagtttatgacatcttcatttttgaggaaacagtgagaccttgcaatctagtggctcaatgtttagcttcccaggacaaaagactcatggctcatggttctacatccccagatacatggtctagtcatggcttctgtgtcctggatgccattttgtcatactttaactcactgattcatggctctgcctgggggcgaaaactatgtgtgaaatccttaagttaatagagaaaataaaagaatcagtacataaatgtaaaagtaaatgaatggaagagaagtgatgcacaggtaatttctgctttcttccatgctccaaatcctcccactgtcaaggagctcaagattttgctgcaatctgactaggtcattgacaaagtaaagataaaaaaaaaatcatgagaaagcccttgggttaaaagccacatcattttccttctacacagtgcattctagtctaacctaaacacgtttaactcatcgcttagagcacaggaagtaacattggtggcccacatgtcaaaaggtggatgtttttgtgactcctggtccttattctccaaagataacaaggtaatgacacaagaaagtttttgttaaagaaattgataaatgtgaagtactgtctgcattttttttattagtccaacttgcatcaaagaggttcataaaataaattggcctcattgagtttatttctggaattatcaattgaacatgtgcttgcaaagacagagacctcttctggccttctctggcttctcattctacagttttcatgagtcctaaaacattttgtctcccatttctccaattaatttcttattcttctcatcacttgccatatttgcacattctgaatattccagttacatactacataccaggcttcatgtttgtcttcccctcttgtatgtgagagcaagggggaaatgaccatgatatgttttgactgaacccctggctcctagaaccatatcgtccatatatatttgttgagtgaatgactagatgcactgcttgaagagtctaacgaaagataaacttagctggataatctgcaaataattatccttgatttttctaatgtaaaatatttgtattaattatagtaatatcactcattatctgcttaccatttgccaggcaatgttgcatgcattttgtacattttgtctcacttaatgctcaatgtgtaggtatgtactattattaaaattatttcacaggcagaggaaccaatgcatagggaagttcaatttcttgcccaagaaatgaagtttaagatgctaagttcaacattctgagtccagagcctgtgctccaggcctctatgaggtaatgttcctcaaaagaaagccatccaggatgggtgggtggctcacagctgtaatcccagcagtttgggaggccaacgtgggtgaattgcttgagtgcagaatatcaaaaacagcctaggaaacatagggagaccttgtctttacaaacataaaaatattatccagccgtggtagcatgtgcttgtgttcccagctacttgggaggcagagctgagaggatcatctgagcctagggaggtggaggctgctgtgagccctgttagtgccactgcactccagcctgtgtgacagaataaaattctgtatcagaaaaagaaaaaagaaaaaacaactgaaaaggaaggacattaaataggaaaagctttaatttatgtgaaaaatgtgttcatttcttgaagtaaaattgcttcacagggacatacatttcccatgactttacctagactgtgaaatgggcccaggagagcagtggttcaaattctctaaaggttgtgttacttttctccatcaagctttctaacgaatgagaaattcttcactatgagtttaattgtacacataccttgtcattcttgtcttattctccaagccccattcccctcctaccctgacttgttcaaagggctggtgaagaaaatttcccaaagatacactctctgttgcagcctacagttcaggaatctgaatcttactctattaaaaaaatgaaaaaataaaaaaagacatgcatatgcatgtgcagtggtttcccaatatgtgtacatgccctgccaaaatgtataaaaggaaggtaaaaagaaaggtgatgcatgctttcagtttcagcttacacaagaagggaggagagaaaagccatggccggtgagtcttttactgtcccaaaaacaggcccctgggaggaaaaagttgtggaaggcgcttgctcttctggaagctatcatcagtatctgtggaggtttatgtgagcaaaagtgagggacagggaggagagtgaggagagaagaaattagatgactcttgcagcctttagaaaggaaaggaaagcccagaaaggaactgaggctttaggagcagcttgctgtcattttgcaatcttatttaaactcttacatgttgtaagtgtggcagaggctacagaattggcacaggaggagtactagcagcagtggttcctaaatgaaatgggctatttctgtttcattactttattattcagagttaaataatcatgtgacataagaggatgatttctgctttccagggactaatgtggaaatggtaaactgagtcttcaggccactccatgcacatgtatggagactattgagggaaataagtagtacagcgataaagtaagaagagggattacccaactgtgaggagggggacatttcactgatgaaatctaaatgaggttgccttctggtgatgacatgagaggaatatgaggctgttttcatgatgtgagaaatccttgtgctccaaacagacaaggtcctgaaggagaagagaaagcagtttatccgttcagtgggcgaaggtacaataaatggcttactgggtgaattattggagacaagggtgctgagccaggaagagatagagatagtaaaatgtgaaaatgctacagttatggataaggcccgagctttgcttgactctgttattcggaaaggggctccagcatgccaaatttgcatcacatacatttgtgaagaagacagtcacctggcagggacgctgggactctcagcaggtaagggtcagtgactcatactcaagttcacctagggcctgtcattgtgactaccatgtgatgtctttactagtcatgttaagttgcagaaaacttccttctctctggcttcaggcaatatttctgtaacacgggccctactgcatgtcccttgactctccttcgtttctgtttactcatcctcagtggccctcttctgattaaagcacatttcctgttctgagtaagcggtagctgatttcctgaaggttatgagaactatctaaaaagcttcacactatatgttccactgttccaggcgaggcacaatatttattgattgatttagtatttttgagtccagggtgtacccaggcacttatgtagttgtctgtcatgaaggtttaaagtctgtaattaaatacgttttataacttctctccaaaatattgccctccaagaagaataagttatatttcaaaagtggtgatatactcagagaatacactgaatgattatagaaaagtaaggatttcaagttggctttttcctcccgttgcctccttgcattttcttttgagtaagatcacccacttgcctgttatcaggctgtggatcatgctgtgagccctggactgcctccatttgtggccaccttgtgacctgatacagtgcacaggaaatgagttggcccattcctgtgatcccttggacctaccacatggtgaggatgagagttctggtggactcctagttactggtctctgtggtactttccaatgccacttcagaggcagatgttctttcaaagtaaaaggctttttttgttttgttttgttttctaagggttagaggaagcttacgcctgtctaagcagacatgggagaagtttatgttttcatctatgtgtaataaatgtcatatttttctgacaatgtctctttcattttatacccccacgactctctaaattaaagaacattttgaacgttatttaatgtcgttcagtcagggttgtgggaggaatctttagtggggagtgttgtaagcattagggtaaagttttccagagatgataagtgactatttcaatgtccttcttctttctctaggtccaacatctggaaatcaccttactacacaagattctcaaatagtacttccttcctaggtaatgctgtttttaaagaaagagcattctttgaaccgtggcttcccgtgacattaatgttgtaggatgaaccacagttaaaggggctatgaagaattcccatagagtgatcatacaattttctttttgtaatctattctgcttttgtagcaactgtcaaaacagcttcactatctatgtttacattaaaatttggaatcagtatctcaaatcctgattacccaaataagtaccaaacttcatcatttaagcctactgaaatattccctcacgtctcctagagacacctttatctaaacaatatcagagctgaacatgccattcttccccaccctgcttctcttccagttttgtgtaactcggttattgatgtcaccatcaccagttgttcaaaccaggaacttgaaacgcatcttggcccctttcctctcacgcatctccaatattcaagccaccgccaagttttatgcattcttctctcaaatttggaactgtctcgctatttcttcttctatttcattaaaaacccctattcatcaagggccctcttaaaataaaagaaacttatatttaaaagcaatttagaggcaagaagtgtaaatttttctatcaacttcagtttttactctattgaatttattaaggttgattaactcatgaatattttcttaaatgttttacagcatgggacactcactatataaccaaatactgcattttcactaagaaaaattacctcaaacataactcatatacctctatttagtttttttatgtcgttttcattcttctttctgaagaaaccaaacatttcttctatcaaattactaatttttagttttctccaatatatccattccaattagttcttccccaagtcaacacaattcatgctgcctttatttatctaactaattgtataacagtttctcaaacttgttttcaaaattaaatattatttccttgacataatagtttctaaaagatactccacaaatctagaaaactaatttctatcttgaaacaaagattacattaagttttttaacaactatatcacattgtcggatggtgttgtactggtgatgaaataaaagctgcaaggattttttcacataaactctgtgaaaacatttcctcatatgtctgtgcagtcaatgttaagtgctataaatccatctagtgtgcacttagattttagcatttatcattctagttcatatatttaataatctccttcttccatatgtatacatagtaggaagagatcctgaaggacaggccaaagttcattaaaaaagtattcctagtgcttactaccatgtctattcagtaaataaataaagagataaacatatttcttaaagatttttggtagattttgttaagacctcctcccattaaattttattctatggttcaaatcttttgaaataaatttatgttctattttatctcacacattggattgttttctgagcattttttcatgcatggccttgaaaagcttcctttctacatttttatctaaaatacttgtaaaaatactgatcctgaaatatgaaaggagatccctgtattgcacacctggaaatctctggctggaaatgtactccattagcagtgaaattattcagcggtgaaagatcaggattgtcttcctaagaatgcatttccacttttgtggcttcatccttaggtgaggctgtaagaaacacaaagcggaacaaagagggatgccaatattcatgatattctttgactcttgcgactcctcaggctgtggcagtgtgggacagccctgctatgcctacatcctcaggctcaggaggaagcatcaagctttgctccctagacgaagctaaaaggatatggaaagaaaagttgacagaggtgccatgctcagaacacaaggttagagtggggtgaaagatacatttgaggcaattctgaaatgcagtggctttttcttagagctgttctcaacggagaatcttacttctgcttcctttacctaaaaaaaaaaaaaaaatcctgcatcctaggatcccaggtttatagaaacagttcctaattgcacccaattcctggataaataacatcaacaatggcagctattgaatgttcactaagagtgggcactgttctaagcattttgtacatgataatacataatttttaaccttaaaaataatccattttagagatctagaagctgagacatggggttgttaaatattctgcccaaagaaaaacaataaaaaattagagcaatcagttttagaattcaggcattctggcttcaaagcttgtgcatttttcaaaatcctacgtatcaagaaaaatgaagtcagtagctaacctgccatctgggcctttgcagtttctatgcctactctcgaagtagcactgaattccgttccccattttcaatgtgtagtccaagggatttggtaccttgataggactaagaaagggaaaatggtcataaattaaccaggtgaatattaaattacagagaataagactattcatccaaactgcatttcctacagatttatccaataatgggcaagtcaagctgcacacatcatgcttgctctcattatctttaacaaagagtttgacagtctttctaaaagagtgggagctgaggttgatgtcataggcatgatgatgctgctacaaaatctggggtacaaggtggatgagaaaagaaatctcactgcttcggtaaggtctattataccatagagacaacggtcatgccctttgtgtaactaaataatattagaaagcttggttgtaggaccaagaattgtaatctgttgataatctaaaatataaacttgccatggaagggtgctggatgctacatattcccttgaaacatcagagtcattcagcatgagtctggtttaacttaaatgtgaaggtttttttttttttttctcagtaatgccacgttctctttaccagataactctgaatgacttcctacaacaccttccttcacctttctcgaaattaagactaaaaggttttttggccctccattgtttgtcatgaaattacttaagaaatccttaagtacttatttattggctttccaaggtgccatgagggttaatcacatagtccaaaatcagcagtatggtctctttcaggaaatgactacagagttggaggtattttctctgcaccaagagcacaaggcctctggtctcatggtattcgggaaggcatttgtgggaagaaatactctgagcaagtcccagatgtattacaactcaatggaatctttaaaacgttgaatagcaagaactgcccaagtttgaaggacaaacccaaggtgataatcatccaggcctgccatgttggtgaggctaatgtctttgagaacaccagacattcaaatactgtcctcatctgtattcatcatgaattttcagtttatgtctgttttttagtgtcaaagaacatacactgtcagcactcaagatgtggcttctgtgaattgtatgactggttggtagatatttgttttatattcaagtctgagactttgatgtttggtggtttttaaaattagtttgagagttagaagaaataagaattgttgaaagaatgttacttgaatgtatgtgtgtatgtgcatgtgaattaatgatagagcaaatatattgataaatatgattgaacagacgctaagaggagaacgttgtcctctcagccattggtagtagtaacaaaccaaattttgtctttccagagaaccatggtgtggtgttggtaaaagattcagtaccggcctctgaaaatatatctttaccaactccgggagattttgaggatgatgctattaatcaagtacacatagagaaggattttattgctttctgctcttccacattaggtaacaggtttcttgtacacagaacatctttgtgggtgttatcaatagaaaaactcacagccacccactagagggtagatacctgtgcagaactttagataagatttttgaacacaaactaaagctttgttccaagttatacacaggattccctttatcctcaaagagcaagtaagcacttcttttcactgatttagtaattaatgaactcctctctgttgcaaaatattttagaactgtgagagataacagagaatgaaatggaagacgatctctgctgtagaacattagagtctgactctacatagaaaaggtcctccatgttgagatcattcaatggaccaattgggcctaacatcaagtattcataggagaagagtagaagcagggttcagaaaagtagaacagcaagcgaggaactttgggcccctaaattaaagtgggttttacctagaagcaagtatataaagcagaatatatacgcattattctaaatgtatgctttgcagtaagaatcttgctattattgacagatctagtcatttagtactttgtaaactagctccactagttcctccctgtactttccttgaattactcactaatatatatgaatataaatatcattttttaaggattaaagaaattaatatatgtgaaatattgcacatgacagaaagaagtacacattaaagaactggtattatttaaaatgtttttattttataacttcattcagttacttttcttagcaaggtttaaataaaaatgacttgaatgcttttatccaccttgtatggcagtttcctgagaggactcgcattgtcttggtcacctctggctcaaaggccctgcctctgctcaagtagttaaattttctgaaataatttgtacaatgagatatcttctttcagaagaagtttggttctatattgtcttgaacatgccctgatgcagaactcaatattttttcaatgatccaaacccatgaccagataaacaccaaaaaaaaatacatttccagcctgagtacatagagtagactataacattagacagaactgttctttaatatcctttgatcccagccttccacatgacattcatggccctctatatagcattttgtaggcccccggcttctcatcaccaatcctgctgatttccgtgtttttcttttctttcattttccttactcctcctttccatatgactgtcagaaactccatttagttatttataactcccatattaagcctagctccacattctgctgtgcaaatcccaactatttcatagtgctctcctgaggaaaagtaactatctacttccttaatttcacaaaacatttgcagaaacacctgtttccaccttatctcttacctacttccttcaactaagatagctgtggttctaaatagaattttattattaatatacactttatgaccattataattggctgaaatgtgtcattaaagaagtctgtgattatacacagagctattgctactgcttttctcttatgcctccctttgtataattttcacatgtcttcattacttccgtgtctaccatgtaataaatattgttttctttaatatttttctcttccataagaacttcttattgtccattatgatgtcaacatccttttattgacttttcctagatttctacttttgttctgttttatgatttttatggttaactcttcgtgtggaaatatcttacagtcaatgttattaaattttcattaaaattttatgtgaagtacatacattgagaatttattaacataattaccctgtatagactgagtattacataccctttgttagctcagacactagtgtagactcagatatctcctgtgtatgaaattgttaccattccctgtatagctgagtctattgcatatcagcatggacaattaacttaaatcacagtcagtaacagcactttggggatgcacatttagatatttctttttcaaatcccaattccttccctttaccacattctgccttttcctaactctctactaaatttacgactgtaactctgggactatgcaacgttagtacctgcaccatatacagcaccatttagagtatcagtaaaaatatactcactgaacttcaaatgtattcactaaaaaatacagtgaagtgtatggaaaatagatgaactccaaaatatgcctcagctggagcagaaatactagagtttcttctctttcttcgggcaagagtcaatcttgagatttaaagctttatcaatagataatgtttgctggagacatcctgcaaagggctctttttattatgagtcattgaacaactgcaagaatattcctgttcctgtgatgtggaggaaattttccacaagatagggctctagtggatgctattttcactctgcttccccgaatgcttggctattgcaattggaagcatagaaagcagatccagacatgtttatcttccaataacacccttccttccaaaaaagttttgaagttctttggagagtgaacaagcctgggagccagatccaatctgttaagaccaacagcctcaataaaataatcatttagtttcacccacctcctgccctgcagagacacatgaatggactctaatttccctgtgtccctcatggacattagatccaaccacagatttaaaacgccctgaggagagtgattcaatgcttagataagcacatggctgtacttgttgtagatacaagtactttttgttttcatcacttactttgctagatttgttttagcctttttggcatgagttaataatcttttcagaaaaacaggtctccagactttcttttctctttcaggttcaactttcatttgagcagccagatgataaagcacagatgcccagcactgaaagagttacttcgacaagacgtttctatctcttcccagtacattaaaataaggaagctgtatgaatgtctgagggccggtatgtgtgtgtggtcaggagtgtgggaaggttgagtaaagtgtactgaaagtccattttagtcaaggactcgaggtctaccgattgagaatttttaatccaaaaatttaaattttgaaatgctctaaaatccaaaactgtgtgagcgcccacatgatattcaaaggaaatgcttattgaagcatttcaaattttaggtttttggaataaggatgctaaaccagtaagtataatgtagatattccaatatacaaaaatatctgaatttcaaaatacttcaggtagcatgaattttggataagggataatcaagccgtggcataatatacagaaaatactgaagtaatgcctttcttctggtctctgcagagcatgctgctcttcttccaaagtttttctttttttcttttagaccctttctgagggtcagctgatattgacctagaagtggccataagtctatgaaggctgactcttagatgtttctccccttcacctcagcagggtaacattttaaaatagcatgtggcctttccttttaaggttgcatatgttcccattggtggtataactttataacttacactctttgctaaaagagaacttttagcaaaattctcttttatgcttgttttttttccccataggaaacctagagtttcttcattctgtaatggtttttggaattatatctgtttaataatagacttctgaacattttaataaatctgatataaaagtaaaaacatgtcctcagttttcttccacattggagcaacagtggttggaaatcagagggagtactgaatttagaatgttagacttaatccgtaagacctgaactggctattggcaccattttttcattcaaaataccatttgctgctggattgaagacacaaattacaaattagggatgcagatacaaattaccacaggtacccgacttcaaaaaatttagagctctgcaggagagatggaaagaagacgaatagttatgacagtgacactgcagtgggataagggacttttcccaaatcccagtttactaccaaaaatccatcaacagaacctgcatatttctaccctttgttaaggtatttaagagagagaaagtaaattttgaattaactgtagaccttcagtagttcacagacttcttgaaaaggtaagacagacatcaaaaatgaagcgcttggcagatggtgcatgacaccctgaagaaactctaggatgcatggtgtgcacagtgaagtccctgcatggcgcaaagaaaatgagttagggctgaaatcactcactcagggatgttaagatttagggaggattggaagaactaccctttcacctgcaaatatgaaccttttctaatctttatcctattaattcaaagtgaatatctgaccttttaaaaaccttcgtagttgagcacattacaatccctttttttgtgtggccttttattaactgtttgtctgggcacgtcttttctgtactattgtgcatattctactacaacctaagactacatctgtgatcagtctttgcgcagagaatatcaaaaagcagaagtaaattgtcaccctcagtaaaaattaattgttaaggcaatcttaagcagttgaaggaaagcattccagggagtaaagggattcctccagagcgctctccaatcaaagggtaagatgacagggagatgtggaggggaagacactgtcatgattcctcagcacagtttaaacggttgcaggtgccaatagtttttgtttttgtcatttatgttgctgtatttgttgcagtttttctggtgtaagattctacatcacaaaaggtccacagactttctcttcttaggtccaattttcatttgagcttccagaggtagggcacagatgctcatcactgagtgacattgacaagatgtttctatctcttcctaggacattaaaagaagtaggttgtatgaatgtccgtgggcaaatgtctgtatgtttgtgttggggatttgaagaagttggagaggagtactgaagctccattctggtcaaggtgtctagttctaaatgtgttactcttcatccaaaatttcaaaatctgaaatcctccagtatctacattctttcctattacctcccacttttttttaatcaaaaagagacactgaaaagtctgcaataattgctagtttaaatgccttcattccattaaattgtaagagtatctcctattgatattgttttttttatataattccaacttttattttccatcaaggggtacatgtgcaggtttgtgacctaggtatattgcatgatgctgtggttttggtgacaattgctcctctcatccaagttgttagcatagtacataatagttagtttctcaacccttgaccctctacctccgtcctccctcttattgaccccagtgtccattgagcccatttgtattctatgtgtacccaatgtgattattcttttgtttctgtacccattaacattccccaaattcccccacgccacactatgcttcccagcctctggaaataattcttcaactctctgtttcattgagttcaaatatttgaaattttagcttccacaaataagtttgatttgcatttcactaataataatgttgtgcattttttcatatacctgtttggcgtttgtatgtactcttttgagaaatgtctatttagatcttctgcccatttttaaatcagattattaaaattttctcctgcagaattgtttgagctccttatatattctggtttttaatcccttgccagatggattgtttgcaattttttgcccattctgtgagttttttctttactttgttgattgtttcctttgctgtgcagaaggtttttaacttgatgtaatcccatttgtccatttttgctttggttgctcatgcttctggagtgttactcaaaaaatctttcttagtccaaaggactggagagtttctctattttttgtaagagttccatagtttgaggtctttgatttaagtctttgattcattttggtatgatttttgtatatggcaggagtcaggggtctaggttcattcttttgcatgtggatatccagtttattttcagtaccatttattaaagagacagtcctctcaccattgtcaaaaatgagttcactgtagatgtgtggatttatctctgggttttgtaatctgttccattgatttatttgtctgtttttataccagtactatgacattttgataattacagttctgcagtgtaacttaaagtcaggtaatgtgattcttacagttttgttatttttcttcagagtacctttggctgttttagatcttttgtggttctatatacattttaggatttctttttattttattatttctgtgaagaatgccaatggtatctttataaggactgcattgaatctatagattgctttaggcagtagagacatgttaacaatattgactcttccaatccataaacaatttttaaaaaatttttgcatcctctttaatatatctttcattaatgttttatagttttcatggcaagggtcttctacttctttggttaatgtaattcctaggtatttaattttaattctagctattgtatatggaataattttcttgatctctttttcagcttgcttaccattggcatatagaaatactattaaattttgcacattaatttttgtatcttgcaatgtttctgaatcaggtctaagagttttttggtggagtctttaggatttttcagatataaggtcacatagtctgcaaacaaggataatttgacttcttcctttccaatctcgatgccctttatttctttatcttttctaattgctctgcctaggacttgcaacactatgttggataacagtgttgaaagtggacatcctgatcttagaggagaggcgttcagttttccccactcagtataatactagctgtggatctgtagtatatgacttttattatgttgaagtatgttctttcagtacctgttgtttttttttgaggattttcatgatgaaggtattttgaattttattaaatgctttttcaacatcaatttaaatgattttatgttttttgtcctttcttctgttgatacaatgtatcacattaattgatttacatatgttgaacattcttgcatcccttggataaatcttacttggtcatgatgaatgatcttttgaatgtgttgttgaatttggcttgctagtgttttgttatggatatttgtatcaatattcatcaatcatactggccgaaattttctttctttttttttttttttgatgtgcctttgtctggttttggtatcagagtaatgctgtccttgtaatgagtttggaagtatttcttcctcctctgttagtctaaggttttttttccttcaaattattttaagttgtggtgtacttgtgtaggatgtgcaggtttgttacataggtaaatgtttgtcttggtggtttgctgtacagattaaccaatcatctaggtattaacccagaatccatcagctattcttcctgatgccctcccttcccctgaccccctcataggccctagcatgtgttgttctctttgctatgtaaatgtgttctcattgttcagttcccactttaagtgagaacatgaggtgtttgcttttctgttcctgcattagtttgttgaggataatggcttccacctccatccatgttcttgggaaggacatgatctgatttctttttatggctgcatagtattccagcatgatgttccatgatgtagctgcacagtattccatgatgtatacataccaccctttcttattcctatctatcactgatgggcatttaggttgattccacgtctttgctattgtgaacagtgctgcagtgaaaatacgcttccatttatctttgtaatagaatgatttatattcttttgggtatgaaaatctccaatctacttcccacagtgctgaactaattcaggtttccaccaatattgtacaggcattgctttctcttcacagtgttgccagcatctgttattttttgacttttattttcacacaaaaaataaaaggagaaaacacttctcaatttattctgtgagaccaatattaacttgatatcagaacttcacaatagcaaagactggaatcaacccaaatgcccatcagcgatagactggataaagaaaatgtggtacatatatgccatttaatactatgcaaccataaagaaggataagatcatgtcctttgcagagacatgaattacctggtagcaattatcctcagaaaactggcacagaaacagaaaaccaaacactgcatgtcctcacttataagtgggagctgaacaatgagaacacatgaacacatggaggagaacaacacacactggggcctgacagggggatttgaagggagggagagcattaggaaaaatatctaatgcatgcttggcttaataactaggtgatagattggtctgtgtagcaaaccaccatggtacacgtttacctaagtcacaaacctgcacatgctgcacattaatcccagaaattaaaaaattatatctaataaattcacaaataaaattatcaaaaattctaaaaaactgaatccagcaactataaaaactattatacatgatgaccaatgatatactatgggtgttttattaatagccattctgattggtgtgacatggtgctttatggtgattttggttcgtttttctctgatgattagtgatgttgaaaattttttcatacattttttgctacttgtttgtcttgttttgagaaatgtttgttcatgtctttcacctactttttaatagggttacttattttttcctgctgagttggtttagttacttacaggttctggatattggacctttgttggatacattgtaaatatgttctctcattctgtcttagcaccatttattgaatagggagtccattccccattgcttatttttgtccacattgtcaaagaaaagatggcatacagctttatttctaagtttttcattttgtctattagtctatgtgtctgtttttgtacgaggaccacactgtttttgttactgtagccttatagtacagattgaagtcaggtaaggtaatgcctccagctttgtttttgttgcttagggtggctttggctattcaggctcttcactggttccacatgaattttagaattgtttttatttttcactctgtgaaaaatggcattaatcatttgataggaataacactgaatctgtggattgccttgggcagatggccattttaatggtattgattcttttactctgtgagcatggaatgttttcccatttgtttgtgtcatacctgattttagtgttttgtagttctccttgtagagatcttttggaatattcctgggtattttcttttccttttttggctactgcaattgaggtgagttcttgatttggctctcagcttcgacagttttggtgtacagaagtgctactgaattttgtacattggaggacagtttgacttcttcttttcctatttggatgccttttatttccttccattgtctgatcactctggctaggacttccaagactatgtggaatagttgtgttgagagagggcatccttgtcttgtgctggttttcaaagggaaatctttcagatgtttggctatgggtttgtcatagttgtctcttattattttggggtctgttccctttgttgcctaatttgttgagggttttttatcataaagggatgttgcattctataaacagcttttctgtgtctgcttttttgttttaaatcctgcttatgtggtgaatcacatttattgacttgtgtatgttgaatcaatcttgcattatgggaatgaagtgcactggattgtggtgagttaactttttgatgtgctgctgtattcagtttactagtattttgttgaacaaaatcgaaaactttttgagtgttgacatgatattcaaaggaaatgcttattgaagtatttctgattttaggttttgggatcaggaatgcccaactagtaagtgtaatgtagatattccaaaattcagaaaagtctgaaatccaaaatattttgtgtccccagcacccagcattttggaaaagagatattctacccatagtataataaacagggaatactgaaagaacaccttccttctgctcagtgcagagtgcactgttcttctctcaaagttcattgcttaaactattccttttgagggtcagttgatgttgatttctgaggtggtaataagatcacaaatgctcacttataggtttttctctctcatttcagccgg

SEQ ID NO: 4 - protein, 110 aa, Homo sapiens
  MISC_FEATURE: first 110 residues of procaspase-1
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSS

SEQ ID NO: 5 - protein, 97 aa, Homo sapiens
  MISC_FEATURE: COP/Pseudo-ICE
MADKVLKEKRKLFIHSMGEGTINGLLDELLQTRVLNQEEMEKVKRENATVMDKTRALIDSVIPKGAQACQICITYICEEDSYLAETLGLSAGPIPGN

SEQ ID NO: 6 - protein, 90 aa, Homo sapiens
  MISC_FEATURE: ICEBERG
MADQLLRKKRRIFIHSVGAGTINALLDCLLEDEVISQEDMNKVRDENDTVMDKARVLIDLVTGKGPKSCCKFIKHLCEEDPQLASKMGLH

SEQ ID NO: 7 - DNA, 42 bp, Artificial Sequence
  INCA-forward primer
cgaggagggatcctagccatggccgacaaggtcctgaaggag

SEQ ID NO: 8 - DNA, 38 bp, Artificial Sequence
  INCA-REVERSE primer
tgaactctcgagaacctaggaaggaagtactatttgag

SEQ ID NO: 9 - DNA, 35 bp, Artificial Sequence
  INCA-specific primer INCA-forward
ggatcctagccatggccgacaaggtcctgaaggag

SEQ ID NO: 10 - DNA, 38 bp, Artificial Sequence; identical to SEQ ID NO: 8
  INCA-specific primer INCA-reverse